(19) United States
(12) Patent Application Publication
(10) Pub. No.: US 2026/0240839 A1
(43) Pub. Date: Aug. 20, 2026

(54) Pharmaceutical composition of tetramethylpyrazine nitrone for injection and a method for manufacture thereof

(71) Applicant: GUANGZHOU MAGPIE PHARMACEUTICALS CO., LTD., Guangzhou (CN)

(72) Inventors: Yuqiang WANG, Guangzhou (CN); Yewei SUN, Guangzhou (CN); Gaoxiao ZHANG, Guangzhou (CN); Zaijun ZHANG, Guangzhou (CN); Peng YI, Guangzhou (CN)

(73) Assignee: GUANGZHOU MAGPIE PHARMACEUTICALS CO., LTD., Guangzhou (CN)

(21) Appl. No.: 18/831,146

(22) PCT Filed: Feb. 24, 2023
(86) PCT No.: PCT/CN2023/000043

§ 371 Date: Aug. 16, 2024

(30) Foreign Application Priority Data
Feb. 25, 2022 (CN) 202210180059.8

(51) Int. Cl. A61K 31/4965; A61K 9/00; A61K 9/19

(52) U.S. Cl. CPC A61K 31/4965; A61K 9/0019; A61K 9/19

(57) ABSTRACT

The present invention provides a pharmaceutical composition of tetramethylpyrazine nitrone for injection and a method of manufacture thereof, wherein the pharmaceutical composition has good stability. The pharmaceutical composition of tetramethylpyrazine nitrone for injection is a freeze-dried powder injection, the characteristics thereof on appearance, acidity, moisture content, related substances, insoluble particles, content detection, sterility, and visible foreign objects are all in compliance with relevant regulations. The method of manufacture of the pharmaceutical composition is simple, and the prepared tetramethylpyrazine nitrone freeze-dried powder injection has good formability. The freeze-drying process of the present invention has good stability and short freeze-drying time, and the process can be performed for effectively saving energy and labor, and is suitable for industrial production.

### FIELD OF THE INVENTION

The present invention relates generally to the field of medicine and, more particularly, to a pharmaceutical preparation of tetramethylpyrazine nitrone, in particular to a pharmaceutical composition of tetramethylpyrazine nitrone for injection and a method for manufacture thereof.

### BACKGROUND OF THE INVENTION

For the treatment of ischemic stroke, there has been no any specific medicine in clinical practice. Most of the medicines in the market fail to meet the requirements due to poor efficacy or significant toxic side effects. The tetramethylpyrazine nitrone compound described herein is a ligustrazine nitrone derivative with a chemical structure of the formula below:

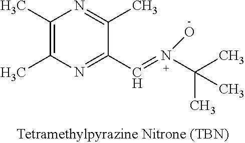

The pharmaceutical preparations used in the treatment of acute ischemic stroke need to have the characteristics of rapid release and the ability of active ingredients to quickly reach the lesion site. Therefore, the most commonly used drug delivery method in clinical practice is intravenous injection. However, there is no any records in the literatures on the pharmaceutical composition of tetramethylpyrazine nitrone for injection.

### SUMMARY OF THE INVENTION

The present invention is directed to provide a pharmaceutical composition of tetramethylpyrazine nitrone for injection and a method for manufacture thereof.

Studies have found that tetramethylpyrazine nitrone has poor stability in some solvents commonly used in clinical practice, for example when placed at room temperature for 6 hours in certain physiological saline or/and glucose injections, the content of related substances significantly increases, thereby affecting the potency and safety of the drug. After a large number of experiments, it was found that the stability of tetramethylpyrazine nitrone can be effectively improved by controlling the pH of the solution of the pharmaceutical composition of tetramethylpyrazine nitrone for injection within a specific range.

In the first aspect, the present invention provides a pharmaceutical composition of tetramethylpyrazine nitrone for injection, wherein the pH of a solvent solution while use of the pharmaceutical composition of tetramethylpyrazine nitrone for injection is maintained between 6.0 and 10.0, preferably between 6.0 and 9.0.

As used herein, the term “solvent solution while use” refers to a solution for clinical rejection that is formed after preparation of the pharmaceutical composition of tetramethylpyrazine nitrone for injection of the present invention with a solvent commonly used in clinical medicine or determined by drug approval for clinical injection. It is within the scope of the present invention when a pharmaceutical composition while use is capable of being dissolved in a solvent commonly used in clinical medicine or determined by drug approval for clinical injection to have pH within the range of 6.0 to 10.0. Prior to clinical use, the pharmaceutical composition of tetramethylpyrazine nitrone for injection may be already in or not in the solvent. In case that the composition is not in solvent, a solvent can be selected according to clinical needs before clinical use.

In some embodiments of the invention, the solvent solution while use of the pharmaceutical composition of tetramethylpyrazine nitrone for injection of the present invention maintains a pH of 6.0 to 10.0 for at least 2 hours, or at least 4 hours, or at least 6 hours.

In some embodiments of the invention, the solvent solution while use of the pharmaceutical composition of tetramethylpyrazine nitrone for injection of the present invention maintains a pH of 6.0 to 10.0, such as 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10.0, or any range formed by any combination of these pH values, such as but not limited to 6.0-9.0, 6.0-8.0, 6.5-9.0, 6.5-8.0, 6.3-9.0, 6.5-7.5, 6.0-8.5.

In some embodiments of the invention, the pharmaceutical composition of tetramethylpyrazine nitrone for injection includes an active ingredient tetramethylpyrazine nitrone and a pH regulator. The pH regulator is selected from pharmaceutically acceptable pH regulators, such as sodium dihydrogen phosphate-sodium hydroxide, sodium sulfite, sodium carbonate, sodium bicarbonate, sodium citrate, sodium hydroxide, potassium dihydrogen phosphate-sodium hydroxide, ammonium hydroxide, concentrated ammonia solution, disodium hydrogen phosphate, sodium acetate, dihydroxymethylaminomethane, ethanolamine, ethylenediamine, alkaline amino acid, borate buffer, and Shackleberry phosphate buffer, sodium acetate borate buffer, or a combination thereof; preferably, in some embodiments, the pH regulator is selected from sodium dihydrogen phosphate-sodium hydroxide, sodium sulfite, sodium bicarbonate, sodium citrate, sodium hydroxide, potassium dihydrogen phosphate-sodium hydroxide, or a combination thereof; more preferably, in some embodiments, the regulator is selected from sodium bicarbonate, sodium citrate, sodium dihydrogen phosphate-sodium hydroxide, or a combination thereof; even more preferably, in some embodiments, the regulator is sodium dihydrogen phosphate-sodium hydroxide.

The dosage range of the pH regulator of the present invention can selected according to the need of maintaining the solvent solution while use with a pH of 6.0 to 10.0, preferably 6.0 to 9.0. In consideration of cost effectiveness, the dosage of the pH regulator can be selected as a lower dosage that can meet the pH conditions. For example, in some exemplary but not limiting embodiments, the weight ratio of tetramethylpyrazine nitrone to the pH regulator can be in any range of 100:(0-50).

In the pharmaceutical composition of tetramethylpyrazine nitrone for injection of the present invention, the weight ratio of tetramethylpyrazine nitrone to the pH regulator is 100:(0-50), for example, 100:0, 100:0.1, 100:0.3, 100:0.5, 100:0.7, 100:1, 100:2, 100:3, 100:4, 100:5, 100:6, 100:8, 100:10, 100:12, 100:15, 100:18, 100:20, 100:25, 100:28, 100:30, 100:32, 100:35, 100:38, 100:40, 100:42, 100:48, or 100:50, or a range of any combination thereof, For example, including but not limited to 100:(0.1-35), 100:(0.3-30), 100:(0.5-25), 100:(1.0-15), 100:(1.5-10).

In some embodiments of the present invention, the pharmaceutical composition of tetramethylpyrazine nitrone for injection includes tetramethylpyrazine nitrone and an excipient; wherein, the excipient is a conventional excipient of pharmaceutical composition for injection, such as sodium chloride, potassium chloride, sodium glutamate, sodium sulfate, sodium lactate, sodium thiosulfate, ammonium acetate, ammonium chloride, sodium bicarbonate, disodium ethylenediaminetetraacetate, sucrose, lactose, maltose, glucose, fructose, trehalose, sorbitol, mannitol, glycerin, inositol, xylitol, citric acid, tartaric acid, amino acid, ethylenediaminetetraacetic acid, skim milk, gelatin, protein, peptone polypeptides, dextrin, serum, methylcellulose, urea, glucan, polyethylene glycol, polyvinylpyrrolidone, vitamin C, vitamin E, thiourea, dimethyl sulfoxide, or a combination thereof. In a specific embodiment, the excipient is mannitol, lactose, maltose, trehalose, sodium chloride, glucose, sorbitol, glycerin, PEG, or propylene glycol, or a combination thereof. In a more specific embodiment, the excipient is mannitol.

In some embodiments of the present invention, in the pharmaceutical composition of tetramethylpyrazine nitrone for injection, the weight ratio of the tetramethylpyrazine nitrone and the excipient is 100:(20-100), preferably 100:(20-60).

In the pharmaceutical composition of tetramethylpyrazine nitrone for injection of the present invention, the weight ratio of tetramethylpyrazine nitrone and the excipient is 100:(20-100), for example 100:20, 100:21, 100:22, 100:23, 100:24, 100:25, 100:26, 100:27, 100:28, 100:29, 100:30, 100:31, 100:32, 100:33, 100:34, 100:35, 100:36, 100:37, 100:38, 100:39, 100:40, 100:41, 100:42, 100:43, 100:44, 100:45, 100:46, 100:47, 100:48, 100:49, 100:50, 100:51, 100:52, 100:53, 100:54, 100:55, 100:56, 100:57, 100:58, 100:59, 100:60, 100:61, 100:62, 100:63, 100:64, 100:65, 100:66, 100:67, 100:68, 100:69, 100:70, 100:71, 100:72, 100:73, 100:74, 100:75, 100:76, 100:77, 100:78, 100:79, 100:80, 100:81, 100:82, 100:83, 100:84, 100:85, 100:86, 100:87, 100:88, 100:89, 100:90, 100:91, 100:92, 100:93, 100:94, 100:95, 100:96, 100:97, 100:98, 100:99, or 100:100, or a range formed with any combination thereof, such as but not limited to 100:(20-60), 100:(20-65), 100:(20-70), 100:(20-75), 100:(20-80), 100:(20-85).

In some embodiments of the present invention, the pharmaceutical composition of tetramethylpyrazine nitrone for injection includes the active ingredient tetramethylpyrazine nitrone and the above-mentioned excipient, and the pharmaceutical composition of tetramethylpyrazine nitrone for injection includes also the pH regulator; i.e., the the pharmaceutical composition of tetramethylpyrazine nitrone for injection includes the active ingredient tetramethylpyrazine nitrone, excipient, and pH regulator.

In some specific embodiments of the present invention, the pharmaceutical composition of tetramethylpyrazine nitrone for injection comprises an active ingredient tetramethylpyrazine nitrone, an excipient, and a pH regulator, wherein the weight ratio of the tetramethylpyrazine nitrone, the excipient, and the pH regulator is 100:(20-100):(0-50), preferrably 100:(20-60):(0.1-35).

In some embodiments of the present invention, the pharmaceutical composition of tetramethylpyrazine nitrone for injection can also include other medicinal auxiliary ingredients for further improving the pharmacological effect or drug stability.

In some embodiments of the present invention, the pharmaceutical composition of tetramethylpyrazine nitrone for injection is an injection agent, a sterile powder agent, or a freeze-dried powder injection agent of tetramethylpyrazine nitrone, preferrably a freeze-dried powder injection agent of tetramethylpyrazine nitrone.

The solvent of the present invention is a commonly used aqueous solvent in the clinical use of medicines, such as water for injection, sterile water for injection, glucose injection, glucose sodium chloride injection, sodium chloride injection, compound sodium chloride injection, sodium lactate Ringer injection, compound sodium lactate glucose injection.

The commonly used aqueous solvents in the clinical use of the present invention, such as water for injection, sterile water for injection, glucose injection, glucose sodium chloride injection, sodium chloride injection, compound sodium chloride injection, sodium lactate Ringer injection, compound sodium lactate glucose injection, all comply with the relevant requirements of the Chinese Pharmacopoeia, 2020 Edition.

In the present invention, the water for injection is distilled from purified water, and the sterilized water for injection is prepared from water for injection according to the injection production process.

The glucose injection described herein is a clinically approved glucose injection, such as a sterilized aqueous solution of glucose or anhydrous glucose, for example, containing glucose or anhydrous glucose in a range of 5% to 50% (g/ml), preferably 5% to 25% (g/ml); or the glucose injection may be of such as 20 ml: 5 g, 20 ml: 10 g, 100 ml: 5 g, 100 ml: 10 g, 250 ml: 12.5 g, 250 ml: 25 g, 500 ml: 25 g, 500 ml: 50 g, or 500 ml: 125 g.

The glucose sodium chloride injection described herein is a clinically approved glucose sodium chloride injection, such as a sterilized aqueous solution of glucose or anhydrous glucose with sodium chloride; for example, a glucose sodium chloride injection of 50 ml: 4 g glucose with 0.09 g sodium chloride, 50 ml: 2.5 g glucose with 0.1 g sodium chloride, or 100 ml: 5 g glucose with 0.2 g sodium chloride, or 100 ml: 2.5 g glucose with 0.45 g sodium chloride, or 100 ml: 5 g glucose with 0.33 g sodium chloride, or 100 ml: 5 g glucose with 0.9 g sodium chloride, or 100 ml: 8 g glucose with 0.18 g sodium chloride, or 100 ml: 10 g glucose with 0.9 g sodium chloride, or 100 ml: 50 g glucose with 9 g sodium chloride, or 250 ml: 12.5 g glucose with 2.25 g sodium chloride, or 250 ml: 12.5 g glucose with 0.5 g sodium chloride, or 250 ml: 6.25 g glucose with 1.125 g sodium chloride, or 250 ml: 20 g glucose with 0.45 g sodium chloride, or 250 ml: 25 g glucose with 2.25 g sodium chloride, or 500 ml: glucose 25 g with 2.25 g sodium chloride, or 500 ml: 12.5 g glucose with 2.25 g sodium chloride, or 500 ml: 25 g glucose with 1.65 g sodium chloride, or 500 ml: 25 g glucose with 4.5 g sodium chloride, or 500 ml: 50 g glucose with 4.5 g sodium chloride.

The sodium chloride injection described herein is a clinically approved sodium chloride injection, such as physiological saline (0.9% sodium chloride injection); preferably a sodium chloride injection of 50 ml: 0.45 mg, 100 ml: 0.9 g, 200 ml: 1.8 g, 250 ml: 2.25 g, 300 ml: 2.7 g, 500 ml: 4.5 mg or 1000 ml: 9 g.

The compound sodium chloride injection described herein is a clinically approved compound sodium chloride injection, such as a sterilized aqueous solution made by mixing sodium chloride, potassium chloride, and calcium chloride; for example, a compound sodium chloride injection containing a total chlorine (Cl) of 0.52% to 0.58% (g/ml), potassium chloride (KCl) of 0.028% to 0.032% (g/ml), and calcium chloride (CaCl₂·2H₂O) of 0.031% to 0.035% (g/ml).

The compound sodium lactate glucose injection described herein is a clinically approved compound sodium lactate glucose injection, such as a sterilized aqueous solution of sodium lactate, sodium chloride, potassium chloride, calcium chloride, and anhydrous glucose.

The sodium lactate Ringer injection described herein is a clinically approved sodium lactate Ringer injection, such as a sterilized aqueous solution of sodium lactate, sodium chloride, potassium chloride, and calcium chloride.

In a second aspect, the present invention also provides a pharmaceutical kit, including the pharmaceutical composition of tetramethylpyrazine nitrone for injection and instructions. In some embodiments, the pharmaceutical kit includes the pharmaceutical composition of tetramethylpyrazine nitrone, instructions, and a solvent of the present invention, wherein the solvent is selected from water for injection, sterilized water for injection, glucose injection, glucose sodium chloride injection, sodium chloride injection, compound sodium chloride injection, sodium lactate Ringer injection, compound sodium lactate and glucose injection, or a combination thereof.

In a third aspect, the present invention also provides a method for manufacture of the pharmaceutical composition of tetramethylpyrazine nitrone for injection.

When the pharmaceutical composition of tetramethylpyrazine nitrone for injection is tetramethylpyrazine nitrone injection or sterile powder of tetramethylpyrazine nitrone, the composition can be prepared via a conventional method in the art. The conventional methods for manufacture are described for example in Pan Weisan, Industrial Pharmacy, China Medical Science and Technology Press, 2010.

The present invention provides a method for manufacture of the pharmaceutical composition of tetramethylpyrazine nitrone for injection, wherein the pharmaceutical composition is a freeze-dried powder injection. The method for manufacture of the pharmaceutical composition of tetramethylpyrazine nitrone for injection comprises: a formula amount of the tetramethylpyrazine nitrone and an excipient were added into water for injection, dissolved, filtered, filled, and then freeze-dried.

In a specific embodiment of the present invention, in the process of preparation, a formula amount of pH regulator is added to water for injection, dissolved, and then a formula amount of excipient and tetramethylpyrazine nitrone are added, dissolved, filtered, filled, and then freeze-dried.

The specific process steps of preparation can be carried out according to conventional methods in this field, which include liquid preparation, filtration, filling with half pressure plug, freeze-drying with full pressure plug, capping, and lamp inspection.

The method of preparation for the tetramethylpyrazine nitrone freeze-dried powder injection described herein can be performed for filtration and filling according to conventional methods in the art, and after freeze-drying, the method can be performed for compressing, discharging, and capping according to conventional methods in the art.

Research has found that there is a material phase separation process in the freeze-drying process for the tetramethylpyrazine nitrone powder injection of the present invention. The occurrence of phase separation causes the solute density at the top of the freeze-dried block to be much higher than the density of other parts. The water vapor flow during sublimation drying is blocked at the top of the freeze-dried block, resulting in appearance defects such as conjunctiva, spray bottle, layering, delamination, and bottom melting in the freeze-dried product. After extensive experimentation, it was found that use of a particular freeze-drying process can improve sublimation efficiency, reduce the probability of appearance defects, and shorten the freeze-drying cycle, and thus effectively saves energy while preparing qualified products of the freeze-dried powder injection of the pharmaceutical composition of tetramethylpyrazine nitrone for injection.

Therefore, in a preferred embodiment of the present invention, the freeze-drying process includes the following steps:
(1) pre freezing: from room temperature to −45° C. in 80 to 180 mins and temperature being maintained for 100-150 mins; from −45° C. to −10° C. in 40-90 mins and temperature being maintained for 100-150 mins; from −10° C. to −45° C. in 40-90 mins and temperature being maintained for 120-400 mins;
(2) primary drying: heating up 10° C. to 30° C. in 60-150 mins, and temperature being maintained for 900-1500 mins, with vacuum being 10-20 Pa;
(3) secondary drying: heating up 30° C. to 60° C. in 40-90 mins, and temperature being maintained for 700-1000 mins, with vacuum being 5-30 Pa.

Preferrably, in the pre freezing stage, “from room temperature to −45° C. in 80 to 180 mins and temperature being maintained for 100-150 mins” is divided into two periods: a first period of “from room temperature to −8 to 10° C. in 40 to 90 mins and temperature being maintained for 50 to 75 mins”; and a second period of “temperature being regulated to −45° C. again in 40 to 90 mins and then temperature being maintained for 50 to 75 mins

The freeze-drying process described herein has good stability, good formability of the freeze-drying preparation, and can expand the pore size of the sublimation channel, reduce sublimation resistance, and shorten the sublimation cycle.

The present invention has the following advantages over the prior art:
(1) The content of the related substances in commonly used clinical solvents increases significantly in a short period of time, which affects the potency and safety of the drug. In the present invention, by controlling the pH of the solvent solution while use of the pharmaceutical composition of tetramethylpyrazine nitrone for injection within a specific range, the stability of tetramethylpyrazine nitrone is effectively improved.
(2) The characteristics on appearance, acidity, moisture content, related substances, insoluble particles, content detection and other indicators of the tetramethylpyrazine nitrone freeze-dried powder injection provided by the present invention are all in compliance with relevant regulations in the field. The results of influencing factor testing, acceleration testing, and long-term testing indicate that the tetramethylpyrazine nitrone freeze-dried powder injection of the present invention has good storage stability and a long drug validity period.
(3) During the freeze-drying of the tetramethylpyrazine nitrone freeze-dried powder injection of the present invention, there are no problems affecting the quality of the freeze-dried powder injection, such as bottle spraying, layering, delamination, and bottom melting. The tetramethylpyrazine nitrone freeze-dried powder injection as prepared has good formability. The freeze-drying process of the present invention has good stability, the prepared crystal form of tetramethylpyrazine nitrone for injection is consistent with the API (Active Pharmaceutical Ingredient), and the freeze-drying time is short, and thus the process can effectively save energy and labor, and is suitable for industrial production.

### DETAILED DESCRIPTION OF THE EMBODIMENTS

Some specific embodiments or examples of the present invention will be described below. It should be pointed out that the following embodiments or examples are only for further explanation of the present invention and cannot be understood as limiting the scope of the invention. Unless otherwise specified, all portions of the present invention are by weight, and all percentages are by mass.

The experimental methods in the following embodiments, unless otherwise specified, are all conventional methods. The experimental materials used in the following embodiments, unless otherwise specified, are all purchased from conventional biochemical reagent suppliers.

### Example 1

The solution stability of tetramethylpyrazine nitrone in different solvents at 25° C. is shown in Table 1.

TABLE 1
Results of Solution Stability at 25° C.

| | sampling time | Content | Total impurities |
|---|---|---|---|
| Medium | (h) | (%) | (%) |
| pH 6.0 phosphate buffer | 0 | 100 | 0.33 |
| | 3 | 98.39 | 0.44 |
| | 6 | 98.65 | 0.9 |
| | 24 | 96.62 | 2.19 |
| Pure water | 0 | 100 | 0.27 |
| | 3 | 99.71 | 0.39 |
| | 24 | 98.54 | 1.16 |
| 0.1M HCl | 0 | NA | 59.28 |
| | 3 | 0 | 100 |
| | 24 | 0 | 100 |
| pH 4.5 Acetate buffer | 0 | NA | 8.36 |
| | 3 | 83.64 | 23.6 |
| | 24 | 60.56 | 44.44 |
| pH 6.4 Phosphate buffer | 0 | 100 | 0.31 |
| | 3 | 98.85 | 0.4 |
| | 24 | 97.78 | 0.93 |
| pH 7.4 Phosphate buffer | 0 | 100 | 0.47 |
| | 3 | 100.83 | 0.39 |
| | 24 | 99.92 | 1 |
| 0.1M NaOH | 0 | NA | 17.78 |
| | 3 | 81.8 | 31.38 |
| | 24 | 56.54 | 49.23 |

Here, “NA” indicates not measured. “Total impurities” at 0 hour refer to that detected when API of tetramethylpyrazine nitrone is just prepared into a solution.

The results in Table 1 indicate that the tetramethylpyrazine nitrone compounds are relatively stable in buffer solutions ranging from pH 6.0 to pH 7.4 at 25° C.; The stability is low in solutions with a pH lower than 6.0 (most commonly used clinical solvents such as physiological saline, glucose injection, and glucose sodium chloride injection have a pH lower than 6.0). In 0.1 M HCl, the stability of tetramethylpyrazine nitrone compounds is low, and they begin to degrade as soon as they form a solution. After 3 hours, they completely degrade, and the total content of related substances reaches 100%.

### Example 2

Prescription composition: Tetramethylpyrazine nitrone 900 g, mannitol 180 g, and water for injection 4500 g; while 9000 bottles were prepared.

Preparation process: In a stainless steel mixing bucket were placed with a precisely measured prescription amount of API and auxiliary materials, a prescription amount of water for injection was added, stirred with a glass rod to be dissolved, and the resulting mixture was filtered using a 0.22 μM filter membrane to remove insoluble substances; the resulting solution was divided into penicillin bottles, and then freeze-dried.

Freeze drying process: Pre freezing period: from room temperature to −45° C. in 150 mins and then the temperature maintained for 120 mins; from −45° C. to −10° C. in 70 mins and then the temperature maintained for 130 mins; from −10° C. to −45° C. in 60 mins and then the temperature maintained for 300 mins. Primary drying: Heating up to 20° C. in 100 mins, and the temperature maintained for 1000 mins with a vacuum of 20 Pa; Secondary drying: Heating up to 50° C. in 70 mins and the temperature maintained for 900 mins with a vacuum of 23 Pa. Then, routine operations were followed for such as pressing plug, capping, light inspection, and product inspection.

The freeze-dried sample prepared has a good morphology and is in a uniform and loose cake shape, without any sample collapse phenomenon. The freeze-dried powder injection prepared in Example 2 was redissolved with 1.6 mL of water for injection, with a pH of 6.4-6.6. See Table 2 for the results of stability testing.

TABLE 2
Results of stability testing on the compatibility
of tetramethylpyrazine nitrone freeze-dried powder
prepared in Example 2 with water for injection

| Testing indicators | 0 hr | 4 hr | 6 hr | 8 hr |
|---|---|---|---|---|
| Acidity (pH) | 6.4 | 6.6 | 6.6 | 6.6 |
| Content % | 100 | 98.9 | 99.3 | 99.8 |
| Pyrazine | 0.04 | 0.09 | 0.11 | 0.13 |
| formaldehyde % | | | | |
| Color of solution | light | light | light | light |
| | yellow | yellow | yellow | yellow |
| Precipitation | not | not | not | not |
| and bubbles | generated | generated | generated | generated |

The results showed that when the sample solution was placed at room temperature for 4 hours, the content of pyrazine formaldehyde was 0.09%, and the content of tetramethylpyrazine nitrone was 98.9%, and the color did not change and there were no bubbles or precipitates generated. After being placed for 6 hours, the pyrazine formaldehyde content was 0.11%; and being placed for 8 hours, the pyrazine formaldehyde content was 0.13%; and the color did not change and there were no bubbles or precipitates generated.

### Example 3

Prescription composition: Tetramethylpyrazine nitrone 700 g, mannitol 264.7 g, sodium dihydrogen phosphate 12.073 g, and NaOH 3.227 g; while 1000 bottles were prepared.

Preparation process: In a solution dispensing tank were placed with water for injection, a prescribed amount of sodium dihydrogen phosphate and sodium hydroxide were added, and stirred until fully dissolved to prepare a buffer solution; to the buffer solution were added a prescribed amount of tetramethylpyrazine nitrone and mannitol, stirred until completely dissolved. According to the filling amount, the resulting solution was filled via weighing method into a 6 mL penicillin bottle, with half stoppers, and marked with the sample number for freeze-drying. Pre freezing period: From room temperature to −45° C. in 180 mins and the temperature maintained for 100 mins; from −45° C. to −10° C. in 90 mins and the temperature maintained for 100 mins; from −10° C. to −45° C. in 90 mins and the temperature maintained for 120 mins. Primary drying: Heating up to 30° C. in 150 mins and the temperature maintained for 900 mins, with vacuum at 10 Pa; Secondary drying: Heating up to 60° C. in 90 mins, and the vacuum of 15 Pa maintained for 700 mins. Then routine operations were followed for such as plug pressing, capping, light inspection, and product inspection.

The freeze-dried sample prepared has a good morphology and is in a uniform and loose cake shape, without any sample collapse phenomenon. The freeze-dried powder injection prepared in Example 3 was redissolved with 250 mL physiological saline, and the acidity, content, related substances, solution color, and other indicators within 24 hours after dissolution were tested. The results are shown in Table 3.

TABLE 3
Quality inspection of tetramethylpyrazine nitrone freeze-dried powder
prepared in Example 3 after resolved in physiological saline.

| Testing indicators | | 0 hr | 6 hr | 24 hr |
|---|---|---|---|---|
| pH | | 7.31 | 7.33 | 7.29 |
| Content | | 98.9% | 98.1% | 98.8% |
| Related | Pyrazine formaldehyde | 0.03% | 0.06% | 0.13% |
| substances | Total impurities | 0.07% | 0.10% | 0.17% |

| | | | |
|---|---|---|---|
| Color of solution | light | light | light |
| | yellow | yellow | yellow |
| Precipitation and bubbles | not | not | not |
| | generated | generated | generated |

The results showed that the sample solution was placed at room temperature for 6 hours, with total impurities of 0.10% and pH of 7.33, and the color did not change and there were no bubbles or precipitates generated; the sample solution was placed at room temperature for 24 hours, with total impurities of 0.17% and pH of 7.29, and the color did not change and there were no bubbles or precipitates generated.

### Examples 4-9

Referring to the method of preparation in Example 3, the effects of different pH regulators on the stability of nitroketosin lyophilized formulations were compared. A solution of freeze-dried powder injection with physiological saline was prepared and placed in a 45° C. constant temperature oven. Samples were taken at 0 and 6 hours and the content of API and total impurities were analyzed. The results are shown in Table 4.

TABLE 4
Quality of tetramethylpyrazine nitrone freeze-dried powder injection of Examples 4-9

| | Example 4 | | Example 5 | | Example 6 | | Example 7 | | Example 8 | | Example 9 | |
|---|---|---|---|---|---|---|---|---|---|---|---|---|
| | Unit | | Unit | | Unit | | Unit | | Unit | | Unit | |
| | dose | Ratio | dose | Ratio | dose | Ratio | dose | Ratio | dose | Ratio | dose | Ratio |
| | (mg) | (%) | (mg) | (%) | (mg) | (%) | (mg) | (%) | (mg) | (%) | (mg) | (%) |
| TBN | 700 | 66.07 | 700 | 80.48 | 700 | 82.64 | 700 | 82.64 | 700 | 82.64 | 700 | 81.81 |
| Mannitol | 140 | 13.21 | 140 | 16.10 | 140 | 16.53 | 140 | 16.53 | 140 | 16.53 | 140 | 16.35 |
| Citric acid | 12.985 | 1.23 | — | — | — | — | — | — | — | — | — | — |
| Na₂HPO₄ | 206.465 | 19.49 | — | — | — | — | — | — | — | — | — | — |
| KH₂PO₄ | — | — | 23.8 | 2.74 | — | — | — | — | — | — | — | — |
| NaOH | — | — | 5.936 + | — | — | — | — | — | — | — | 2.03 | 0.24 |
| | | | q.s. | | | | | | | | | |
| NaHCO₃ | — | — | — | — | 7 | 0.83 | — | — | — | — | — | — |
| Sodium | — | — | — | — | — | — | 7 | 0.83 | — | — | — | — |
| citrate | | | | | | | | | | | | |
| Na₂SO₃ | — | — | — | — | — | — | — | — | 7 | 0.83 | — | — |
| NaH₂PO₄ | — | — | — | — | — | — | — | — | — | — | 13.65 | 1.60 |
| Purified water | 3500 | N/A | 3500 | N/A | 3500 | N/A | 3500 | N/A | 3500 | N/A | 3500 | N/A |

| | | | | | | |
|---|---|---|---|---|---|---|
| pH of solution | 7.01 | 8.6 | 8.46 | 7.72 | 9.02 | 7.41 |

Stability results

| Content | 0 h | 100 | 100.53 | 99.07 | 100.16 | 97.77 | 103.36 |
|---|---|---|---|---|---|---|---|
| (%) | 6 h | 99.57 | 99.02 | 98.19 | 99.82 | 92.28 | 101.23 |
| Total | 0 h | 0.00 | 0.00 | 0.08 | 0.16 | 0.27 | 0 |
| impurities | 6 h | 0.43 | 0.14 | 0.23 | 0.28 | 0.66 | 0.12 |
| (%) | | | | | | | |

Note:
An impurity content of 0.00 indicates that it is below the detection limit and has not been detected.

Table 4 shows that when the pH regulator is citric acid disodium hydrogen phosphate, sodium bicarbonate, sodium citrate, sodium sulfite, potassium dihydrogen phosphate sodium hydroxide, or sodium dihydrogen phosphate sodium hydroxide, the tetramethylpyrazine nitrone solution has good stability.

### Examples 10-16

### Investigation on the Effects of the Amounts of Excipients

Referring to the method of preparation in Example 3, the effects of the dosage of excipients on the freeze-dried powder injection of tetramethylpyrazine nitrone was compared, and the results are shown in Table 5

TABLE 5
Formulation of tetramethylpyrazine nitrone freeze-dried powder injection of Example 10-16

| | Example 10 | | Example 11 | | Example 12 | | Example 13 | | Example 14 | | Example 15 | | Example 16 | |
|---|---|---|---|---|---|---|---|---|---|---|---|---|---|---|
| | (20 bottle | | (20 bottle | | (20 bottle | | (20 bottle | | (20 bottle | | (20 bottle | | (20 bottle | |
| Component | quantity) | | quantity) | | quantity) | | quantity) | | quantity) | | quantity) | | quantity) | |
| TBN | 14.0000 | g | 14.0000 | g | 14.0000 | g | 14.0000 | g | 14.0000 | g | 140.0000 | g | 140.0000 | g |
| Mannitol | 1.6625 | g | 2.4625 | g | 4.2600 | g | 5.2647 | g | 6.2600 | g | 83.9400 | g | 140.0000 | g |
| Sodium | 0.2730 | g | 0.2730 | g | 0.2730 | g | 0.2730 | g | 0.2730 | g | 2.4146 | g | 2.730 | g |
| dihydrogen | | | | | | | | | | | | | | |
| phosphate | | | | | | | | | | | | | | |
| Sodium | 0.0645 | g | 0.0645 | g | 0.0645 | g | 0.0645 | g | 0.0645 | g | 0.6454 | g | 0.645 | g |
| hydroxide | | | | | | | | | | | | | | |
| Water for | 70.0000 | g | 70.0000 | g | 70.0000 | g | 70.0000 | g | 70.0000 | g | 700.0000 | g | 700.0000 | g |
| injection | | | | | | | | | | | | | | |
| Filling | 4.30 | g/btl | 4.34 | g/btl | 4.43 | g/btl | 4.48 | g/btl | 4.53 | g/btl | 4.64 | g/btl | 4.92 | g/btl |
| capacity | | | | | | | | | | | | | | |

| | | | | | | | |
|---|---|---|---|---|---|---|---|
| Appearance | There are | There are | The | The | The | The | The |
| | huge voids | huge voids | structure is | structure is | structure is | structure is | structure is |
| | in the | in the | dense and | dense and | dense and | dense and | dense and |
| | internal | internal | complete, | complete, | complete, | complete, | complete, |
| | structure, | structure, | with no | with no | with no | with no | with no |
| | and the | and the | obvious | obvious | obvious | obvious | obvious |
| | freeze-dried | freeze-dried | voids. | voids. | voids. | voids. | voids. |
| | powder is | powder is | | | | | |
| | sticky. | sticky. | | | | | |

Table 5 shows that when the weight ratio of mannitol to tetramethylpyrazine nitrone is low, such as when the weight ratio is (11.875-17.589):100 (Examples 10-11), tetramethylpyrazine nitrone freeze-dried powder has poor appearance with huge voids in the internal structure and sticky freeze-dried powder in the penicillin bottle after opening. On the other hand, when the weight ratio of mannitol to tetramethylpyrazine nitrone is within the range of the invention (Examples 12-16), the freeze-dried powder has a dense and complete structure with no obvious voids.

### Examples 17-20

Example 17 (quantity of 20 bottles): 14.0000 g of tetramethylpyrazine nitrone, 5.5617 g of lactose, 0.2732 g of sodium dihydrogen phosphate, 0.0655 g of sodium hydroxide, and 70.000 g of water for injection; while the filling amount is 4.495 g.
Example 18 (quantity of 20 bottles): 14.0000 g of tetramethylpyrazine nitrone, 5.5725 g of glucose, 0.2732 g of sodium dihydrogen phosphate, 0.0655 g of sodium hydroxide, and 70.000 g of water for injection; while the filling amount is 4.496 g.
Example 19 (quantity of 20 bottles): 14.0000 g of tetramethylpyrazine nitrone, 4.9523 g of glycerol, 0.2732 g of sodium dihydrogen phosphate, 0.0655 g of sodium hydroxide, and 70.000 g of water for injection; while the filling amount is 4.465 g.
Example 20 (amount of 20 bottles): 14.0000 g of tetramethylpyrazine nitrone, 6.2635 g of maltose, 0.2732 g of sodium dihydrogen phosphate, 0.0655 g of sodium hydroxide, and 70.000 g of water for injection; while the filling amount is 4.530 g.

By using the method of preparation of Example 3, the freeze-dried powders of Example 17-20 were prepared, and the appearance of the freeze-dried products was compared. The products in Example 17-20 appeared to have a dense and complete structure with no obvious voids, good sample formability, and no appearance defects such as bottle spraying, layering, delamination, and bottom melting.

### Examples 21-30

### Effects of Different pH Phosphate Buffers on the Stability of Tetramethylpyrazine Nitrone Solution

A buffer solution of potassium dihydrogen phosphate sodium hydroxide with a pH of 6.6~9.0 was prepared, prescribed amounts of the drugs were added separately, and the prepared solution was placed in a 45° C. constant temperature oven. Samples were taken and analyzed at 0 and 6 hours, respectively. The experimental prescription and results are shown in Table 6.

TABLE 6
Investigation of the effect of different buffer pH on the stability of tetramethylpyrazine nitrone solution

| | Example 21 | | Example 22 | | Example 23 | | Example 24 | | Example 25 | |
|---|---|---|---|---|---|---|---|---|---|---|
| | Unit | | Unit | | Unit | | Unit | | Unit | |
| | dose | Ratio | dose | Ratio | dose | Ratio | dose | Ratio | dose | Ratio |
| | (mg) | (%) | (mg) | (%) | (mg) | (%) | (mg) | (%) | (mg) | (%) |
| TBN | 700 | 80.66 | 700 | 80.53 | 700 | 80.48 | 700 | 80.48 | 700 | 80.48 |
| Mannitol | 140 | 16.13 | 140 | 16.10 | 140 | 16.10 | 140 | 16.10 | 140 | 16.10 |
| Potassium | 23.8 | 2.74 | 23.8 | 2.74 | 23.8 | 2.74 | 23.8 | 2.74 | 23.8 | 2.74 |
| dihydrogen | | | | | | | | | | |
| phosphate | | | | | | | | | | |
| Sodium hydroxide | 4.074 | 0.47 | 5.474 | 0.63 | 5.936 | 0.68 | 5.936 + | — | 5.936 + | — |
| | | | | | | | q.s. | | q.s. | |
| Purified water | 3500 | N/A | 3500 | N/A | 3500 | N/A | 3500 | N/A | 3500 | N/A |

| | | | | | |
|---|---|---|---|---|---|
| Solution pH | 6.6 | 6.8 | 7.0 | 7.8 | 8.0 |

Stability results

| Content | 0 h | 101.12 | 100.07 | 100.79 | 100.67 | 100.81 |
|---|---|---|---|---|---|---|
| (%) | 6 h | 99.73 | 99.85 | 100.00 | 100.00 | 99.81 |
| Total | 0 h | 0.00 | 0.00 | 0.00 | 0.00 | 0.00 |
| impurities | 6 h | 0.27 | 0.15 | 0.00 | 0.00 | 0.19 |
| (%) | | | | | | |

| | | Example 26 | | Example 27 | | Example 28 | | Example 29 | | Example 30 | |
|---|---|---|---|---|---|---|---|---|---|---|---|
| | | Unit | | Unit | | Unit | | Unit | | Unit | |
| | | dose | Ratio | dose | Ratio | dose | Ratio | dose | Ratio | dose | Ratio |
| | | (mg) | (%) | (mg) | (%) | (mg) | (%) | (mg) | (%) | (mg) | (%) |
| TBN | | 700 | 80.48 | 700 | 80.48 | 700 | 80.48 | 700 | 80.48 | 700 | 80.48 |
| Mannitol | | 140 | 16.10 | 140 | 16.10 | 140 | 16.10 | 140 | 16.10 | 140 | 16.10 |
| Potassium | | 23.8 | 2.74 | 23.8 | 2.74 | 23.8 | 2.74 | 23.8 | 2.74 | 23.8 | 2.74 |
| dihydrogen | | | | | | | | | | | |
| phosphate | | | | | | | | | | | |
| Sodium hydroxide | | 5.936 + | — | 5.936 + | — | 5.936 + | — | 5.936 + | — | 5.936 + | — |
| | | q.s. | | q.s. | | q.s. | | q.s. | | q.s. | |
| Purified water | | 3500 | N/A | 3500 | N/A | 3500 | N/A | 3500 | N/A | 3500 | N/A |
| Solution pH | | 8.2 | | 8.4 | | 8.6 | | 8.8 | | 9.0 | |
| Stability results | | | | | | | | | | | |
| Content | 0 h | 100.30 | | 99.95 | | 100.53 | | 99.81 | | 99.96 | |
| (%) | 6 h | 96.91 | | 98.47 | | 99.02 | | 99.35 | | 98.23 | |
| Total | 0 h | 0.00 | | 0.00 | | 0.00 | | 0.00 | | 0.00 | |
| impurities | 6 h | 0.15 | | 0.14 | | 0.14 | | 0.13 | | 0.13 | |
| (%) | | | | | | | | | | | |

Note:
An impurity content of 0.00 indicates that it is below the detection limit and has not been detected. Example 28 in Table 6 and Example 5 in Table 4 are of the same experiment.

In Table 6, “q.s.” refers that, in addition of the amount of sodium hydroxide added in Example 23, an appropriate amount of sodium hydroxide was added to adjust the tetramethylpyrazine nitrone solution to a specified pH value.

The results show a good stability within the pH range of 6.6~9.0.

### Example 31

### 1. Preparation of Tetramethylpyrazine Nitrone Freeze-Dried Powder Injection

### (1) Prescription Composition:

Tetramethylpyrazine nitrone 5600.0000 g, mannitol 2117.6000 g, sodium dihydrogen phosphate 96.5840 g, sodium hydroxide 25.8160 g, and water for injection 28000.0000 g; while 8000 bottles were prepared.

### (2) Preparation Process:

In a solution dispensing tank were placed with water for injection, a prescribed amount of sodium dihydrogen phosphate and sodium hydroxide were added, and stirred until fully dissolved to prepare a buffer solution; to the buffer solution were added a prescribed amount of tetramethylpyrazine nitrone and mannitol and stirred until completely dissolved; According to the filling amount, the resulting solution was filled via weighing method into a 6 mL penicillin bottle, with half stoppers, and marked the sample number for freeze-drying. Pre freezing period: The temperature of the imported heat transfer oil was controlled at a constant rate to reach −9° C. within 60 mins and the temperature maintained for 60 mins; the temperature of the imported heat transfer oil was controlled at a constant speed to reach −45° C. within 60 mins and the temperature maintained for 60 mins; the temperature of imported heat transfer oil was controlled at a constant speed to reach −10° C. within 60 mins and the temperature maintained for 120 mins; the temperature of the imported heat transfer oil was controlled at a constant speed to reach −45° C. within 60 mins and the temperature maintained for 240 mins. Water-trapping refrigeration: The water trap was turned on and cooled, and when the temperature of the water trap reaches below −45° C., the vacuum pump was started. The vacuum of the drying oven reached below 50 Pa, and heating sublimation started. Sublimation drying: The inlet temperature of the heat transfer oil was controlled at a constant rate to reach 20° C. within 90 mins and the temperature maintained for 1080 mins, and the pressure was set to 15 Pa. After the sublimation drying was completed, heating was continued to enter the step of desorption drying. Desorption drying: The inlet temperature of the heat transfer oil was controlled at a constant rate to reach 50° C. within 60 mins and the temperature maintained for 840 mins, and the pressure was set to 10 Pa.

2. Compatibility Stability of the Tetramethylpyrazine Nitrone Freeze-Dried Powder Injection with Physiological Saline:

The inventors investigated the compatibility stability after the freeze-dried powder injection prepared in Example 31 was redissolved with physiological saline (Guangdong Kelun). The stabilities of the solutions in 3 different concentration gradients (7 mg/ml, 14 mg/ml, and 28 mg/ml) at room temperature for 8 hours are shown in Table 7. The results showed that the key quality attributes such as acidity, content, pyrazine formaldehyde, and total impurities of the sample solution with different concentration gradients after being redissolved with physiological saline at room temperature for 8 hours all met the quality standards.

TABLE 7
Compatability stabilities of the of TBN for injection with physiological saline

| | | Physiological saline supplier | | | | | | | | |
|---|---|---|---|---|---|---|---|---|---|---|
| | | Guangdong Kelun (pH: 5.51) | | | | | | | | |
| | | Liquid concentration | | | | | | | | |
| | | 7 mg/ml | | | 14 mg/ml | | | 28 mg/ml | | |
| Testing indicators | | 0 hr | 4 hr | 8 hr | 0 hr | 4 hr | 8 hr | 0 hr | 4 hr | 8 hr |
| Acidity | | 7.45 | 7.22 | 7.14 | 7.56 | 7.37 | 7.3 | 7.58 | 7.49 | 7.44 |
| Content (%) | | 96.02 | 95.24 | 95.30 | 95.85 | 94.86 | 94.70 | 96.48 | 96.42 | 95.14 |
| Related | Pyrazine | 0.03 | 0.04 | 0.06 | 0.03 | 0.04 | 0.06 | 0.02 | 0.04 | 0.05 |
| substances | formaldehyde (%) | | | | | | | | | |
| | Total impurities (%) | 0.04 | 0.05 | 0.07 | 0.04 | 0.05 | 0.07 | 0.03 | 0.05 | 0.06 |

### 3. Study on Stability of the Preparation of the Tetramethylpyrazine Nitrone Freeze-Dried Powder Injection

In order to test the stability of the drug formulation containing tetramethylpyrazine nitrone prepared by the present invention, the freeze-dried powder injection product of tetramethylpyrazine nitrone prepared in Example 31 was taken and redissolved with water for injection to run an influence factor test on high temperature, high humidity and strong light irradiation, accelerated stability, and long-term stability.

### (1) High-Temperature Test

A high-temperature test was run in accordance with the guidelines for tests on stability of API and pharmaceutical formulations (General Rules <9001>, Chinese Pharmacopoeia, 2015 Edition) and relevant provisions of ICH Q1A. An appropriate amount of the sample from Example 31 was taken and placed at a temperature of 60±2° C. for a long period of 10 days, and samples were taken on the day 5 and day 10 respectively, dissolved with water for injection, to test the relevant substances and content using a high-performance liquid chromatography (General Rule <0512>, Part 4, Chinese Pharmacopoeia, 2015 Edition). The test results were compared with that of the sample of day 0 for the items of stability test which include appearance, moisture, related substances, insoluble particles, and content inspection. The test results are shown in Table 8.

TABLE 8
Results of high-temperature test (Example 31)

| | | Test conditions | | |
|---|---|---|---|---|
| | | High temperature test 60 ± 2° C. | | |
| Time (day) | | 0 | 5 | 10 |
| Apparence | | White | Light | Light |
| | | solid | yellow | yellow |
| | | | solid | solid |
| Moisture content (%) | | 0.62 | 0.48 | 0.54 |
| Related | Pyrazine | below the | 0.20 | 0.24 |
| substances | formaldehyde | detection limit | | |
| (%) | Total impurities | 0.02 | 0.34 | 0.40 |
| Insoluble | ≥10 μm | 21 | NA | 410.8 |
| particles | ≥25 μm | 0 | NA | 0 |

| | | | |
|---|---|---|---|
| Content (%) | 100.2 | 100.4 | 98.3 |

Note:
“NA” indicates not measured.

### (2) High-Humidity Test

A high-humidity test was run in accordance with the guidelines for tests on stability of API and pharmaceutical formulations (General Rules <9001>, Chinese Pharmacopoeia, 2015 Edition) and relevant provisions of ICH Q1A. An appropriate amount of the sample from Example 31 was taken and placed at a temperature of 25±2° C. and relative humidity of 90±10% for a long period of 10 days, and samples were taken on the day 5 and day 10 respectively, dissolved with water for injection to test the relevant substances and content using a high-performance liquid chromatography (General Rule <0512>, Part 4, Chinese Pharmacopoeia, 2015 Edition). The test results were compared with that of the sample of day 0 for the items of stability test which include appearance, moisture, related substances, insoluble particles, and content inspection. The test results are shown in Table 9.

TABLE 9
Results of high-humidity test (Example 31)

| | | Test conditions | | |
|---|---|---|---|---|
| | | High humidity test 25 ± | | |
| | | 2° C./90 ± 10% RH | | |
| Time (day) | | 0 | 5 | 10 |
| Apparence | | White | White | White |
| | | solid | solid | solid |
| Moisture content (%) | | 0.62 | 0.59 | 0.60 |
| Related | Pyrazine | below the | 0.01 | 0.01 |
| substances | formaldehyde | detection | | |
| (%) | | limit | | |
| | Total impurities | 0.02 | 0.03 | 0.03 |
| Insoluble | ≥10 μm | 21 | NA | 185 |
| particles | ≥25 μm | 0 | NA | 0 |

| | | | |
|---|---|---|---|
| Content (%) | 100.2 | 101.3 | 100.5 |

Note:
“NA” indicates not measured.

### (3) Strong Light Irradiation Test

A strong light irradiation test was run in accordance with the guidelines for tests on stability of API and pharmaceutical formulations (General Rules <9001>, Chinese Pharmacopoeia, 2015 Edition) and relevant provisions of ICH Q1A. An appropriate amount of the sample from Example 31 was taken and placed under illumination of 4500±500 Lx for 10 days, and samples were taken on the day 5 and day 10 respectively, dissolved with water for injection to test the relevant substances and content using a high-performance liquid chromatography (General Rule <0512>, Part 4, Chinese Pharmacopoeia, 2015 Edition). The test results were compared with that of the sample of day 0 for the items of stability test which include appearance, moisture, related substances, insoluble particles, and content inspection. The test results are shown in Table 10.

TABLE 10
Results of strong light irradiation test (Example 31)

| | | Test conditions | | |
|---|---|---|---|---|
| | | Strong light irradiation 4500 ± 500 Lx | | |
| Time (day) | | 0 | 5 | 10 |
| Apparence | | White | White | White |
| | | solid | solid | solid |
| Moisture content (%) | | 0.62 | 0.49 | 0.60 |
| Related | Pyrazine | below the | 0.01 | 0.01 |
| substances | formaldehyde | detection limit | | |
| (%) | Total impurities | 0.02 | 0.03 | 0.03 |
| Insoluble | ≥10 μm | 21 | NA | 133.2 |
| particles | ≥25 μm | 0 | NA | 0 |

| | | | |
|---|---|---|---|
| Content determination (%) | 100.2 | 101.1 | 101.3 |

Note:
“NA” indicates not measured.

### (4) Accelerated Stability Test

An accelerated stability test was run in accordance with the guidelines for tests on stability of API and pharmaceutical formulations (General Rules <9001>, Chinese Pharmacopoeia, 2015 Edition) and relevant provisions of ICH Q1A, for exploring the stability of drugs by accelerating their chemical or physical changes. An appropriate amount of the sample from Example 31 was taken- and accelerated for 6 months at a temperature of 40±2° C. and a relative humidity of 75±5%, and samples were taken at the end of month 0, 1, 2, 3, and 6, respectively, dissolved with water for injection to test the relevant substances and content using a high-performance liquid chromatography (General Rule <0512>, Part 4, Chinese Pharmacopoeia, 2015 Edition). The test was performed to check the key items of stability test, which include appearance, moisture, related substances, insoluble particles, and content inspection. Testing results are shown in Table 11.

TABLE 11
Results of acceleration test (Example 31)

| | | Experimental conditions | | | | |
|---|---|---|---|---|---|---|
| | | Accelerated testing, 40 ± 2° C./75 ± 5% RH | | | | |
| | | Time (month) | | | | |
| | | 0 | 1 | 2 | 3 | 6 |
| Forms | | white | white | white | white | white |
| | | solid | solid | solid | solid | solid |
| Moisture (%) | | 0.62 | 0.61 | 0.52 | 0.43 | 0.56 |
| Related | Pyrazine | below | 0.01 | below | 0.01 | 0.01 |
| substance | formaldehyde | detection | | detection | | |
| (%) | | limit | | limit | | |
| | Total impurities | 0.02 | 0.03 | 0.02 | 0.04 | 0.03 |
| Insoluble | ≥10 μm | 21 | 32 | 35 | 266 | 382.5 |
| particles | ≥25 μm | 0 | 0 | 0 | 0 | 0 |

| | | | | | |
|---|---|---|---|---|---|
| Content detection (%) | 100.2 | 101.9 | 101.2 | 97.3 | 99.1 |

### (5) Long Term Test

The long-term testing was conducted in accordance with the guidelines for tests on stability of API and pharmaceutical formulations (General Rule <9001>, Chinese Pharmacopoeia, 2015 Edition) and relevant provisions of ICH Q1A. The experiment was conducted under conditions close to the storage of pharmaceutical reagents, with the aim of providing a basis for determining the expiration date of the drug. An appropriate amount of the sample from Example 31 and stored for 18 months at a temperature of 25±2° C. and a relative humidity of 60±5%. Samples were taken respectively at time points 0, 3, 6, 9, 12, and 18 months, re-dissolved with water for injection, and the relevant substances and content were determined by using high-performance liquid chromatography (General Rule <0512>, Part 4, Chinese Pharmacopoeia, 2015 Edition). Testing was taken on major items of stability, including appearance, moisture, related substances, insoluble particles, and content detection. The test results are shown in Table 12.

TABLE 12
Results of long-term test (Example 31)

| | | Test conditions | | | | | |
|---|---|---|---|---|---|---|---|
| | | Long-term test, 25 ± 2° C./60 ± 5% RH | | | | | |
| | | Time (month) | | | | | |
| | | 0 | 3 | 6 | 9 | 12 | 18 |
| Appearance | | white | white | white | white | white | white |
| | | solid | solid | solid | solid | solid | solid |
| Moisture (%) | | 0.62 | 0.58 | 0.57 | 0.42 | 0.44 | 0.36 |
| Related | Pyrazine | below | 0.01 | 0.01 | 0.01 | 0.01 | 0.01 |
| substances | formaldehyde | detection | | | | | |
| (%) | | limit | | | | | |
| | Total impurities | 0.02 | 0.03 | 0.03 | 0.03 | 0.03 | 0.03 |
| Insoluble | ≥10 μm | 21 | 119 | 39 | 180 | 66.5 | 330.75 |
| particles | ≥25 μm | 0 | 0 | 0 | 0 | 0 | 1.5 |

| | | | | | | |
|---|---|---|---|---|---|---|
| Content inspection (%) | 100.2 | 98.6 | 100.1 | 101.5 | 100.0 | 98.6 |

According to the investigation results of the influencing factors (under high temperature, high humidity, and strong light irradiation conditions), the products are relatively stable for high humidity and strong light irradiation. After 6 months of accelerated testing and 18 months of long-term testing, as compared to day 0, the products meets the requirements in terms of Appearance, moisture, related substances, insoluble particles, and content detection.

### (6) Investigation on the Stability of Crystal Form

The present invention has determined the crystal form of API, accessory materials, and prepared injection sample powder of tetramethylpyrazine nitrone for preparation of the pharmaceutical composition of tetramethylpyrazine nitrone for injection. The results show that the crystal form of the prepared injection tetramethylpyrazine nitrone is consistent with that of the raw material medicine, indicating that the production process of the formulation has not changed the crystal form of the raw material medicine.

### Examples 32-34

According to the formulation of Example 31, the freeze-dried formulations of Examples 32-34 were processed via the freeze-dried process shown in Table 13 to investigate the effect of the freeze-dried process on the freeze-dried products.

TABLE 13
Effect of freeze-drying process on freeze-dried products

| | Example 32 | Example 33 | Example 34 |
|---|---|---|---|
| Process | Parameters of the freeze-drying process | | |
| Enter the box | Products enterring at room temperature (RT) | | |
| Pre-freezing | Atmospheric pressure: | Atmospheric pressure: | RT→ −45° C. in 80 mins, |
| | RT→ −35° C. in 120 mins, and | RT→ −5° C. in 60 mins, and | and termperature |
| | termperature maintained for | termperature maintained for | maintained for 150 mins; −45° |
| | 300 mins. | 120 mins; −5° C.→ −40° C. in | C.→ −10° C. in 40 |
| | | 60 mins, and termperature | mins, and termperature |
| | | maintained for 120 mins. | maintained for 150 mins; −10° |
| | | | C→ −45° C. in 40 |
| | | | mins, and termperature |
| | | | maintained for 400 mins. |
| First drying | Vacuum: 10 ± 5 Pa, −35° | Vacuum: 10 ± 5 Pa, −40° | Vacuum: 20 Pa, the |
| | C. → −25° C. in 120 mins, | C. → −10° C. in 30 mins, | temperature raised to 10° C. |
| | and termperature maintained | termperature maintained for | in 60 mins, and |
| | for 780 mins. | 900 mins. | termperature maintained |
| | | | for 1500 mins. |
| Second | Vacuum: 10 ± 5 Pa, −25° C. | Vacuum: 10 ± 5 Pa, −10° C. | Vacuum degree is 25 Pa, |
| drying | to −5° C. in 180 mins, and | to 10° C. in 120 mins; and | temperature raised to 30° C. |
| | termperature maintained for | termperature maintained for | in 40 mins, and |
| | 120 mins; −5° C. to 10° C. in 60 | 60 mins; | termperature maintained |
| | mins; termperature | 10° C. to 25° C. in 30 mins; | for 1000 mins. |
| | maintained for 60 mins; | and termperature maintained | |
| | 10° C. to 25° C. in 30 mins, | for 720 mins. | |
| | and termperature maintained | Extreme vacuum, maintained | |
| | for 720 mins. | for 180 mins. | |
| | Extreme vacuum, maintained | | |
| | for 180 mins. | | |
| Appearance | Severely delaminated, and | The upper surface is slightly | The freeze-dried sample |
| of | the freeze-dried products | collapsed, and some samples | has a good morphology and |
| freeze-dried | shrink. | have bottom melting | is in a uniform and loose |
| products | | phenomenon. | cake shape, with no |
| | | | evidence of sample |
| | | | collapse. |

The inventor investigated the effects of different freeze-drying processes on freeze-dried products, and the results showed that the freeze-dried products prepared by the freeze-drying process in Examples 32-33 showed severe delamination, shrinkage of the freeze-dried products, slight surface collapse of the freeze-dried products, and some samples had appearance defects such as bottom melting. The freeze-dried product prepared in Example 34 showed no any appearance defects such as conjunctiva, spray bottle, delamination, delamination, and bottom melting during the freeze-dried process; the prepared freeze-dried products had a good morphology and were in a uniform and loose cake shape.

1. A pharmaceutical composition of tetramethylpyrazine nitrone for injection, wherein the pH of a solvent solution while use of the pharmaceutical composition of tetramethylpyrazine nitrone for injection is maintained between 6.0 and 10.0.

2. The pharmaceutical composition of claim 1, wherein the pharmaceutical composition of tetramethylpyrazine nitrone for injection comprises an active ingredient tetramethylpyrazine nitrone and a pH regulator;
wherein, the pH regulator is selected from sodium dihydrogen phosphate-sodium hydroxide, sodium sulfite, sodium carbonate, sodium bicarbonate, sodium citrate, sodium hydroxide, potassium dihydrogen phosphate-sodium hydroxide, ammonium hydroxide, concentrated ammonia solution, disodium hydrogen phosphate, sodium acetate, dihydroxymethylaminomethane, ethanolamine, ethylenediamine, alkaline amino acid, borate buffer, Shackleberry phosphate buffer, sodium acetate borate buffer, or a combination thereof.

3. The pharmaceutical composition of claim 1 or 2, wherein the pharmaceutical composition of tetramethylpyrazine nitrone for injection comprises an active ingredient tetramethylpyrazine nitrone and an excipient;
wherein, the excipient is selected from sodium chloride, potassium chloride, sodium glutamate, sodium sulfate, sodium lactate, sodium thiosulfate, ammonium acetate, ammonium chloride, sodium bicarbonate, disodium ethylenediaminetetraacetate, sucrose, lactose, maltose, glucose, fructose, trehalose, sorbitol, mannitol, glycerin, inositol, xylitol, citric acid, tartaric acid, amino acid, ethylenediaminetetraacetic acid, skimmed milk, gelatin, protein, peptone polypeptide, dextrin, serum, methylcellulose, urea, glucan, polyethylene glycol, polyvinyl pyrrolidone, vitamin C, vitamin E, thiourea, dimethyl sulfoxide, or a combination thereof.

4. The pharmaceutical composition of claim 3, wherein the weight ratio of the tetramethylpyrazine nitrone and the excipient is 100:(20-100); preferrably 100:(20-60).

5. The pharmaceutical composition of claim 1, wherein the pharmaceutical composition of tetramethylpyrazine nitrone for injection comprises an active ingredient tetramethylpyrazine nitrone, an excipient, and a pH regulator; wherein the weight ratio of the tetramethylpyrazine nitrone, the excipient, and the pH regulator is 100:(20-100):(0-50).

6. The pharmaceutical composition of claim 1, wherein the pharmaceutical composition of tetramethylpyrazine nitrone for injection is an injection agent of tetramethylpyrazine nitrone, a sterile powder agent of tetramethylpyrazine nitrone, or a freeze-dried powder injection agent of tetramethylpyrazine nitrone.

7. The pharmaceutical composition of claim 1, wherein the solvent is water for injection, sterile water for injection, glucose injection, glucose sodium chloride injection, sodium chloride injection, compound sodium chloride injection, sodium lactate Ringer injection, or compound sodium lactate glucose injection.

8. The pharmaceutical composition of claim 7, wherein:
the glucose injection is a sterilized aqueous solution of glucose or anhydrous glucose, preferably comprising 5% to 50% (g/ml) of glucose or anhydrous glucose, and more preferably comprising 5% to 25% (g/ml) of glucose or anhydrous glucose, or for example of 20 ml: 5 g, 20 ml: 10 g, 100 ml: 5 g, 100 ml: 10 g, 250 ml: 12.5 g, 250 ml: 25 g, 500 ml: 25 g, 500 ml: 50 g, or 500 ml: 125 g;
the glucose sodium chloride injection is a sterilized aqueous solution of glucose or anhydrous glucose with sodium chloride, preferably the glucose sodium chloride injection comprising 50 ml: 4 g glucose with 0.09 g sodium chloride, 50 ml: 2.5 g glucose with 0.1 g sodium chloride, or 100 ml: 5 g glucose with 0.2 g sodium chloride, or 100 ml: 2.5 g glucose with 0.45 g sodium chloride, or 100 ml: 5 g glucose with 0.33 g sodium chloride, or 100 ml: 5 g glucose with 0.9 g sodium chloride, or 100 ml: 8 g glucose with 0.18 g sodium chloride, or 100 ml: 10 g glucose with 0.9 g sodium chloride, or 100 ml: 50 g glucose with 9 g sodium chloride, or 250 ml: 12.5 g glucose with 2.25 g sodium chloride, or 250 ml: 12.5 g glucose with 0.5 g sodium chloride, or 250 ml: 6.25 g glucose with 1.125 g sodium chloride, or 250 ml: 20 g glucose with 0.45 g sodium chloride, or 250 ml: 25 g glucose with 2.25 g sodium chloride, or 500 ml: glucose 25 g with 2.25 g sodium chloride, or 500 ml: 12.5 g glucose with 2.25 g sodium chloride, or 500 ml: 25 g glucose with 1.65 g sodium chloride, or 500 ml: 25 g glucose with 4.5 g sodium chloride, or 500 ml: 50 g glucose with 4.5 g sodium chloride;
the sodium chloride injection is a physiological saline, preferrably 0.9% sodium chloride injection, or for example of 50 ml: 0.45 mg, 100 ml: 0.9 g, 200 ml: 1.8 g, 250 ml: 2.25 g, 300 ml: 2.7 g, 500 ml: 4.5 mg, or 1000 ml: 9 g;
the compound sodium chloride injection is a sterilized aqueous solution made by mixing sodium chloride, potassium chloride, and calcium chloride, preferably containing a total chlorine (Cl) of 0.52% to 0.58% (g/ml), potassium chloride (KCl) of 0.028% to 0.032% (g/ml), and calcium chloride (CaCl₂·2H₂O) of 0.031% to 0.035% (g/ml);
the compound sodium lactate glucose injection is a sterilized aqueous solution of sodium lactate, sodium chloride, potassium chloride, calcium chloride, and anhydrous glucose;
the sodium lactate Ringer injection is a sterilized aqueous solution of sodium lactate, sodium chloride, potassium chloride, and calcium chloride.

9. A pharmaceutical kit, comprising:
the pharmaceutical composition of tetramethylpyrazine nitrone for injection of claim 1 and instructions; or
the pharmaceutical composition of tetramethylpyrazine nitrone for injection of claim 1, instructions, and a solvent, wherein the solvent is selected from water for injection, sterile water for injection, sodium chloride injection, glucose injection, glucose sodium chloride injection, compound sodium chloride injection, sodium lactate Ringer injection, compound sodium lactate and glucose injection, or a combination thereof.

10. A method for manufacture of the pharmaceutical composition of tetramethylpyrazine nitrone for injection of claim 1, wherein the pharmaceutical composition is a freeze-dried powder injection;
wherein the method for manufacture of the freeze-dried powder injection comprises: a formula amount of the tetramethylpyrazine nitrone and an excipient were added into water for injection, dissolved, filtered, filled in bottles, and then freeze-dried; preferably, a pH regulator was added into the water for injection, dissolved, and an excipient and the tetramethylpyrazine nitrone were added, dissolved, filtered, filled in bottles, and freeze-dried.

11. The method of claim 10, wherein the step of freeze-drying comprises the following stages:
(1) pre freezing: from room temperature to −45° C. in 80 to 180 mins and temperature being maintained for 100-150 mins; from −45° C. to −10° C. in 40-90 mins and temperature being maintained for 100-150 mins; from −10° C. to −45° C. in 40-90 mins and temperature being maintained for 120-400 mins;
(2) primary drying: heating up 10° C. to 30° C. in 60-150 mins, and temperature being maintained for 900-1500 mins, with vacuum being 10-20 Pa;
(3) secondary drying: heating up 30° C. to 60° C. in 40-90 mins, and temperature being maintained for 700-1000 mins, with vacuum being 5-30 Pa;
preferrably, in the pre freezing stage, “from room temperature to −45° C. in 80 to 180 mins and temperature being maintained for 100-150 mins” is divided into two periods: a first period of “from room temperature to −8 to 10° C. in 40 to 90 mins and temperature being maintained for 50 to 75 mins”; and a second period of “temperature being regulated to −45° C. again in 40 to 90 mins and then temperature being maintained for 50 to 75 mins.

12. The pharmaceutical composition of claim 2, wherein the pH of a solvent solution while use of the pharmaceutical composition of tetramethylpyrazine nitrone for injection is maintained between 6.0 and 9.0.

13. The pharmaceutical composition of claim 1, wherein the pH regulator is selected from sodium dihydrogen phosphate-sodium hydroxide, sodium sulfite, sodium bicarbonate, sodium citrate, sodium hydroxide, potassium dihydrogen phosphate-sodium hydroxide, or a combination thereof.

14. The pharmaceutical composition of claim 13, wherein the pH regulator is selected from sodium bicarbonate, sodium citrate, sodium dihydrogen phosphate-sodium hydroxide, or a combination thereof.

15. The pharmaceutical composition of claim 14, wherein the pH regulator is sodium dihydrogen phosphate-sodium hydroxide.

16. The pharmaceutical composition of claim 3, wherein the excipient is selected from mannitol, lactose, maltose, trehalose, sodium chloride, glucose, sorbitol, glycerin, PEG, propylene glycol, or a combination thereof.

17. The pharmaceutical composition of claim 16, wherein the excipient is mannitol.

18. The pharmaceutical composition of claim 5, wherein the weight ratio of the tetramethylpyrazine nitrone, the excipient, and the pH regulator is 100:(20-60):(0.1-35).

19. The pharmaceutical composition of claim 6, wherein the pharmaceutical composition of tetramethylpyrazine nitrone for injection is a freeze-dried powder injection agent of tetramethylpyrazine nitrone.
